(19) United States
(12) Patent Application Publication
(10) Pub. No.: US 2024/0112786 A1
(43) Pub. Date: Apr. 4, 2024

(54) IMAGE PROCESSING APPARATUS, IMAGE PROCESSING METHOD, AND IMAGE PROCESSING PROGRAM

(71) Applicant: FUJIFILM Corporation, Tokyo (JP)

(72) Inventors: Aya OGASAWARA, Tokyo (JP); Mizuki TAKEI, Tokyo (JP)

(73) Assignee: FUJIFILM Corporation, Tokyo (JP)

(21) Appl. No.: 18/528,754

(22) Filed: Dec. 4, 2023

Related U.S. Application Data
(63) Continuation of application No. PCT/JP2022/018960, filed on Apr. 26, 2022.

(30) Foreign Application Priority Data
Jun. 25, 2021 (JP) 2021-105656

(51) Int. Cl. G16H 30/40; G06T 7/00

(52) U.S. Cl. CPC G16H 30/40; G06T 7/0012; G06T 2207/10081

(57) ABSTRACT

A processor is configured to extract a target organ from a medical image, set a plurality of small regions in the target organ, derive a first evaluation value representing a physical quantity of each of the plurality of small regions, derive at least one second evaluation value representing a relationship between the first evaluation values in the plurality of small regions, and derive a third evaluation value indicating presence or absence of an abnormality in the entire target organ based on the second evaluation value.

### CROSS-REFERENCE TO RELATED APPLICATIONS

This application is a continuation of International Application No. PCT/JP2022/018960, filed on Apr. 26, 2022, which claims priority from Japanese Patent Application No. 2021-105656, filed on Jun. 25, 2021. The entire disclosure of each of the above applications is incorporated herein by reference.

### BACKGROUND

### Technical Field

The present disclosure relates to an image processing apparatus, an image processing method, and an image processing program.

### Related Art

In recent years, with the progress of medical devices, such as a computed tomography (CT) apparatus and a magnetic resonance imaging (MRI) apparatus, it is possible to make an image diagnosis by using a medical image having a higher quality and a higher resolution. In addition, computer-aided diagnosis (CAD), in which the presence probability, positional information, and the like of a lesion are derived by analyzing the medical image and presented to a doctor, such as an image interpretation doctor, is put into practical use.

However, in some cases, the lesion is not clearly shown on the medical image depending on a type and a size of the lesion or a method of capturing the medical image. For example, a tumor related to pancreatic cancer is relatively clearly shown in a contrast tomographic image of an abdomen, but the tumor related to the pancreatic cancer is hardly shown in a non-contrast tomographic image. Since the CAD in the related art is developed on the premise that the lesion is clearly shown on the medical image to some extent, it is difficult to find the lesion that is hardly shown as described above.

Therefore, a method has been proposed in which a target organ is divided into small regions and a diagnosis is made using evaluation results of the small regions. For example, WO2010-035517A proposes a method of obtaining an intercostal region in the lung, calculating a feature amount representing the size of the intercostal region, and determining an abnormality based on a difference between the feature amounts of the corresponding intercostal regions in the left and right lungs. In addition, “Liu, Kao-Lang, et al. “Deep learning to distinguish pancreatic cancer tissue from non-cancerous pancreatic tissue: a retrospective study with cross-racial external validation.” The Lancet Digital Health 2.6 (2020): e303-e313” proposes a method of determining normal or abnormal of the entire target organ by dividing the target organ into small regions, calculating an evaluation value representing the presence or absence of lesions in each small region, and integrating the evaluation values.

However, in the method described in WO2010-035517A, only the presence or absence of the lesion in a partial region of the target organ can be determined. In addition, since the shape change of the target organ is not localized in the small region, it is not possible to accurately determine the shape change of the small region only by integrating the evaluation values of the small region as described in the literature of Liu et al.

The present disclosure has been made in view of the above circumstances, and an object of the present disclosure is to enable accurate evaluation of an abnormality of a target organ in consideration of a relationship between a plurality of small regions in the target organ.

### SUMMARY OF THE INVENTION

The present disclosure relates to an image processing apparatus comprising at least one processor,
in which the processor is configured to:
extract a target organ from a medical image;
set a plurality of small regions in the target organ;
derive a first evaluation value representing a physical quantity of each of the plurality of small regions;
derive at least one second evaluation value representing a relationship between the first evaluation values in the plurality of small regions; and
derive a third evaluation value indicating presence or absence of an abnormality in the entire target organ based on the second evaluation value.

It should be noted that, in the image processing apparatus according to the present disclosure, the first evaluation value may be a physical quantity related to a size of the small region, and the second evaluation value may be an evaluation value related to a difference in sizes between the small regions.

In addition, in the image processing apparatus according to the present disclosure, the processor may be configured to set an axis passing through the target organ, and set the small region along the axis.

In addition, in the image processing apparatus according to the present disclosure, the processor may be configured to display an evaluation result based on the third evaluation value on a display.

In addition, in the image processing apparatus according to the present disclosure, the evaluation result based on the third evaluation value may be an occurrence probability of a finding representing a feature of a shape of the target organ.

In addition, in the image processing apparatus according to the present disclosure, the finding may include at least one of atrophy, swelling, stenosis, or dilation that occurs in the target organ.

In addition, in the image processing apparatus according to the present disclosure, the processor may be configured to display a position of the small region having a relatively high contribution to the evaluation result in the target organ on the display as distinguished from a position of the small region having a relatively low contribution.

In addition, in the image processing apparatus according to the present disclosure, the processor may be configured to display at least one of the first evaluation value or the second evaluation value of the small region having the relatively high contribution to the evaluation result in the target organ on the display.

In addition, in the image processing apparatus according to the present disclosure, the medical image may be a tomographic image of an abdomen including a pancreas, and the target organ may be a pancreas.

In addition, in the image processing apparatus according to the present disclosure, the processor may be configured to set the small region by dividing the pancreas into a head portion, a body portion, and a caudal portion.

The present disclosure relates to an image processing method comprising:
extracting a target organ from a medical image;
setting a plurality of small regions in the target organ;
deriving a first evaluation value representing a physical quantity of each of the plurality of small regions;
deriving at least one second evaluation value representing a relationship between the first evaluation values in the plurality of small regions; and
deriving a third evaluation value indicating presence or absence of an abnormality in the entire target organ based on the second evaluation value.

It should be noted that a program for causing a computer to execute the image processing method according to the present disclosure may be provided.

According to the present disclosure, it is possible to accurately evaluate the abnormality of the target organ.

### BRIEF DESCRIPTION OF THE DRAWINGS

FIG. 1 is a diagram illustrating a schematic configuration of a diagnosis support system to which an image processing apparatus according to an embodiment of the present disclosure is applied.

FIG. 2 is a diagram illustrating a hardware configuration of the image processing apparatus according to the present embodiment.

FIG. 3 is a functional configuration diagram of the image processing apparatus according to the present embodiment.

FIG. 4 is a diagram illustrating extraction of a pancreas from a target image.

FIG. 5 is a diagram illustrating a setting of a small region.

FIG. 6 is a diagram illustrating a setting of the small region.

FIG. 7 is a diagram illustrating a setting of the small region.

FIG. 8 is a diagram illustrating derivation of a diameter in a cross section of the pancreas.

FIG. 9 is a diagram illustrating a schematic configuration of a recurrent neural network.

FIG. 10 is a diagram illustrating a table in which an age and a threshold value are defined.

FIG. 11 is a diagram illustrating an evaluation result display screen.

FIG. 12 is a diagram illustrating an evaluation result display screen.

FIG. 13 is a diagram illustrating an evaluation result display screen.

FIG. 14 is a flowchart illustrating processing performed in the present embodiment.

FIG. 15 is a diagram illustrating a main pancreatic duct and a pancreas parenchyma.

FIG. 16 is a diagram schematically illustrating a cross section of the pancreas.

### DETAILED DESCRIPTION

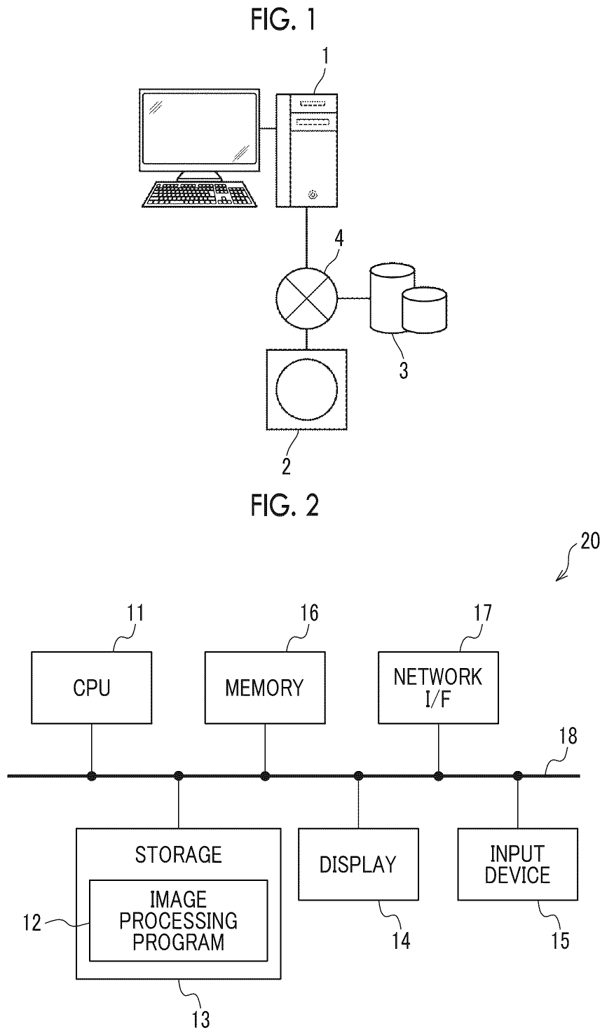

In the following, embodiments of the present disclosure will be explained with reference to the drawings. First, a configuration of a medical information system to which an image processing apparatus according to the present embodiment is applied will be described. FIG. 1 is a diagram illustrating a schematic configuration of the medical information system. In the medical information system illustrated in FIG. 1, a computer 1 including the image processing apparatus according to the present embodiment, an imaging apparatus 2, and an image storage server 3 are connected via a network 4 in a communicable state.

The computer 1 includes the image processing apparatus according to the present embodiment, and an image processing program according to the present embodiment is installed in the computer 1. The computer 1 may be a workstation or a personal computer directly operated by a doctor who makes a diagnosis, or may be a server computer connected to the workstation or the personal computer via the network. The image processing program is stored in a storage device of the server computer connected to the network or in a network storage to be accessible from the outside, and is downloaded and installed in the computer 1 used by the doctor, in response to a request. Alternatively, the image processing program is distributed in a state of being recorded on a recording medium, such as a digital versatile disc (DVD) or a compact disc read only memory (CD-ROM), and is installed in the computer 1 from the recording medium.

The imaging apparatus 2 is an apparatus that images a diagnosis target part of a subject to generate a three-dimensional image showing the part and is, specifically, a CT apparatus, an MRI apparatus, a positron emission tomography (PET) apparatus, and the like. The three-dimensional image consisting of a plurality of tomographic images generated by the imaging apparatus 2 is transmitted to and stored in the image storage server 3. It should be noted that, in the present embodiment, the imaging apparatus 2 is a CT apparatus, and a CT image of a thoracoabdominal portion of the subject is generated as the three-dimensional image. It should be noted that the acquired CT image may be a contrast CT image or a non-contrast CT image.

The image storage server 3 is a computer that stores and manages various types of data, and comprises a large-capacity external storage device and database management software. The image storage server 3 communicates with another device via the wired or wireless network 4, and transmits and receives image data and the like to and from the other device. Specifically, the image storage server 3 acquires various types of data including the image data of the three-dimensional image generated by the imaging apparatus 2 via the network, and stores and manages the various types of data in the recording medium, such as the large-capacity external storage device. It should be noted that the storage format of the image data and the communication between the devices via the network 4 are based on a protocol, such as digital imaging and communication in medicine (DICOM).

Next, the image processing apparatus according to the present embodiment will be described. FIG. 2 is a diagram illustrating a hardware configuration of the image processing apparatus according to the present embodiment. As illustrated in FIG. 2, the image processing apparatus 20 includes a central processing unit (CPU) 11, a non-volatile storage 13, and a memory 16 as a transitory storage region. In addition, the image processing apparatus 20 includes a display 14, such as a liquid crystal display, an input device 15, such as a keyboard and a mouse, and a network interface (I/F) 17 connected to the network 4. The CPU 11, the storage 13, the display 14, the input device 15, the memory 16, and the network I/F 17 are connected to a bus 18. It should be noted that the CPU 11 is an example of a processor according to the present disclosure.

The storage 13 is realized by a hard disk drive (HDD), a solid state drive (SSD), a flash memory, and the like. An image processing program 12 is stored in the storage 13 as a storage medium. The CPU 11 reads out the image processing program 12 from the storage 13, develops the image processing program 12 in the memory 16, and executes the developed image processing program 12.

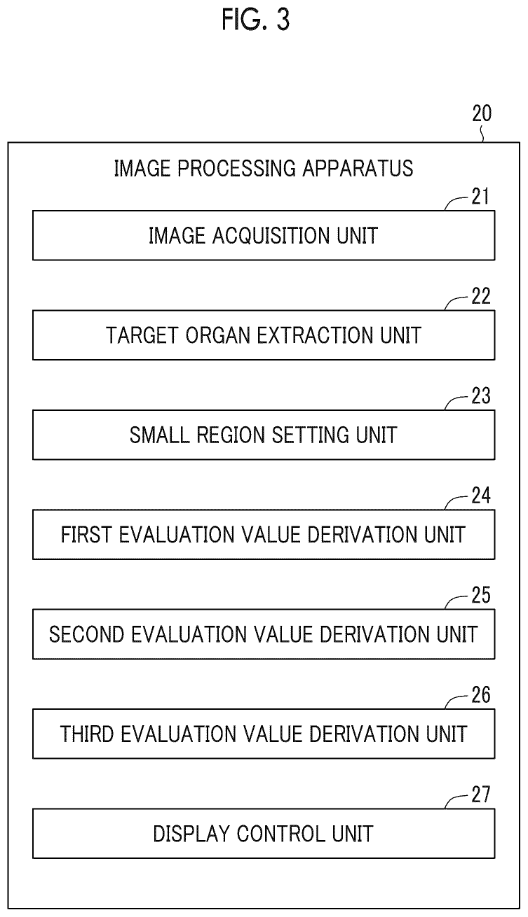

Hereinafter, a functional configuration of the image processing apparatus according to the present embodiment will be described. FIG. 3 is a diagram illustrating the functional configuration of the image processing apparatus according to the present embodiment. As illustrated in FIG. 3, the image processing apparatus 20 comprises an image acquisition unit 21, a target organ extraction unit 22, a small region setting unit 23, a first evaluation value derivation unit 24, a second evaluation value derivation unit 25, a third evaluation value derivation unit 26, and a display control unit 27. By executing the image processing program 12 by the CPU 11, the CPU 11 functions as the image acquisition unit 21, the target organ extraction unit 22, the small region setting unit 23, the first evaluation value derivation unit 24, the second evaluation value derivation unit 25, the third evaluation value derivation unit 26, and the display control unit 27.

The image acquisition unit 21 acquires a target image G0 that is a processing target from the image storage server 3 in response to an instruction from the input device 15 by an operator. In the present embodiment, the target image G0 is the CT image including the plurality of tomographic images including the thoracoabdominal portion of the human body as described above. The target image G0 is an example of a medical image according to the present disclosure.

The target organ extraction unit 22 extracts the target organ from the target image G0. In the present embodiment, the target organ is a pancreas. The target organ extraction unit 22 includes a semantic segmentation model (hereinafter, referred to as a SS model) subjected to machine learning to extract the pancreas from the target image G0. As is well known, the SS model is a machine learning model that outputs an output image in which a label representing an extraction object (class) is assigned to each pixel of the input image. In the present embodiment, the input image is a tomographic image constituting the target image G0, the extraction object is the pancreas, and the output image is an image in which a region of the pancreas is labeled. The SS model is constructed by a convolutional neural network (CNN), such as residual networks (ResNet) or U-shaped networks (U-Net).

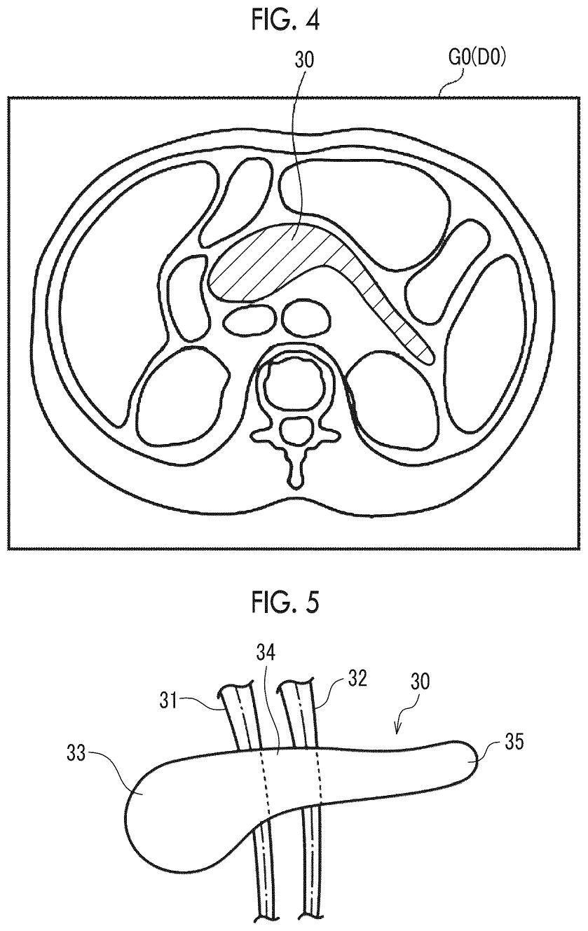

As illustrated in FIG. 4, a pancreas 30 is extracted from the target image G0 by the target organ extraction unit 22. It should be noted that FIG. 4 illustrates one tomographic image D0 included in the target image G0.

The extraction of the target organ is not limited to the extraction using the SS model. Any method of extracting the target organ from the target image G0, such as template matching or threshold value processing, can be applied.

The small region setting unit 23 sets a plurality of small regions in the pancreas which is the target organ extracted from the target image G0 by the target organ extraction unit 22. In the present embodiment, the small region setting unit 23 sets each of a head portion, a body portion, and a caudal portion as a small region by dividing the pancreas 30 extracted from the target image G0 into the head portion, the body portion, and the caudal portion.

FIG. 5 is a diagram illustrating the division of the pancreas into the head portion, the body portion, and the caudal portion. It should be noted that FIG. 5 is a diagram of the pancreas as viewed from the front of the human body. In the following description, the terms “up”, “down”, “left”, and “right” are based on a case in which the human body in a standing posture is viewed in the front. As illustrated in FIG. 5, in a case in which the human body is viewed from the front, a vein 31 and an artery 32 run in parallel in the up-down direction at an interval behind the pancreas 30. The pancreas 30 is anatomically divided into a head portion on the left side of the vein 31, a body portion between the vein 31 and the artery 32, and a caudal portion on the right side of the artery 32. Therefore, in the present embodiment, the small region setting unit 23 divides the pancreas 30 into three small regions of the head portion 33, the body portion 34, and the caudal portion 35, with reference to the vein 31 and the artery 32. It should be noted that boundaries of the head portion 33, the body portion 34, and the caudal portion 35 are based on the boundary definition described in “General Rules for the Study of Pancreatic cancer 7th Edition, Revised and Enlarged Version, edited by Japan Pancreas Society, page 12, September, 2020”. Specifically, a left edge of the vein 31 (a right edge of the vein 31 in a case in which the human body is viewed from the front) is defined as a boundary between the head portion 33 and the body portion 34, and a left edge of the artery 32 (a right edge of the artery 32 in a case in which the human body is viewed from the front) is defined as a boundary between the body portion 34 and the caudal portion 35.

For division, the small region setting unit 23 extracts the vein 31 and the artery 32 in the vicinity of the pancreas 30 in the target image G0. The small region setting unit 23 extracts a blood vessel region and a centerline (that is, the central axis) of the blood vessel region from the region in the vicinity of the pancreas 30 in the target image G0, for example, by the method described in JP2010-200925A and JP2010-220732A. In this method, first, positions of a plurality of candidate points constituting the centerline of the blood vessel and a principal axis direction are calculated based on values of voxel data constituting the target image G0. Alternatively, positional information of the plurality of candidate points constituting the centerline of the blood vessel and the principal axis direction are calculated by calculating the Hessian matrix for the target image G0 and analyzing eigenvalue of the calculated Hessian matrix. Then, a feature amount representing the blood vessel likeness is calculated for the voxel data around the candidate point, and it is determined whether or not the voxel data represents the blood vessel based on the calculated feature amount. Accordingly, the blood vessel region and the centerline thereof are extracted from the target image G0. The small region setting unit 23 divides the pancreas 30 into the head portion 33, the body portion 34, and the caudal portion 35, with reference to the left edge of the extracted vein 31 and artery 32 (a right edge in a case in which the human body is viewed from the front).

It should be noted that the division of the pancreas 30 into the head portion 33, the body portion 34, and the caudal portion 35 is not limited to the method described above. For example, the pancreas 30 may be divided into the head portion 33, the body portion 34, and the caudal portion 35 by using the segmentation model subjected to machine learning to extract the head portion 33, the body portion 34, and the caudal portion 35 from the pancreas 30. In this case, the segmentation model may be trained by preparing a plurality of pieces of teacher data consisting of pairs of a teacher image including the pancreas and a mask image obtained by dividing the pancreas into the head portion, the body portion, and the caudal portion based on the boundary definitions described above.

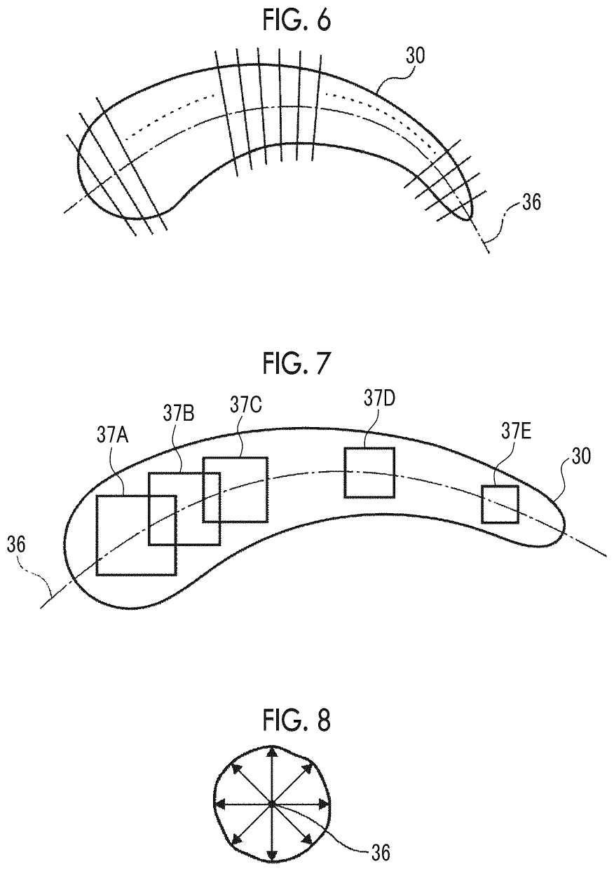

In addition, the setting of the small region for the pancreas 30 is not limited to the division into the head portion 33, the body portion 34, and the caudal portion 35. FIG. 6 is a diagram illustrating another example of a small region setting. It should be noted that FIG. 6 is a diagram of the pancreas 30 as viewed from a head portion side of the human body. In the other example, the small region setting unit 23 extracts a central axis 36 extending in a longitudinal direction of the pancreas 30. As a method of extracting the central axis 36, the same method as the above-described method of extracting the centerlines of the vein 31 and the artery 32 can be used. Then, the small region setting unit 23 may set small regions in the pancreas 30 by dividing the pancreas 30 into a plurality of small regions at equal intervals along the central axis 36.

Further, as illustrated in FIG. 7, small regions 37A to 37C that overlap each other may be set in the pancreas 30, or small regions spaced from each other, such as small regions 37D and 37E, may be set. In this case, the small region may be set along the central axis 36 of the pancreas 30 or may be set at any position.

The first evaluation value derivation unit 24 derives a first evaluation value representing a physical quantity of each of the plurality of small regions set by the small region setting unit 23. In the present embodiment, the physical quantity related to a size of the small region is derived as the first evaluation value. Specifically, the representative value of the diameters of the head portion 33, the body portion 34, and the caudal portion 35 of the pancreas 30 are derived as the first evaluation values E11, E12, and E13, respectively.

The first evaluation value derivation unit 24 first sets the central axis 36 of the pancreas 30 as illustrated in FIG. 6. Then, in each of the head portion 33, the body portion 34, and the caudal portion 35, a plurality of cross sections orthogonal to the central axis 36 are set at equal intervals along the central axis 36. Here, the cross section orthogonal to the central axis 36 of the pancreas 30 has an oval shape as illustrated in FIG. 8, but is not a perfect circle. For this reason, the first evaluation value derivation unit 24 derives lengths of the cross section orthogonal to the central axis 36 in a plurality of directions (for example, four directions of the up-down direction, the left-right direction, the lower right to upper left direction, and the lower left to upper right direction) passing through the central axis 36, and derives the representative value of the lengths in the plurality of directions as the diameter of the cross section. For example, an average value, a median value, a minimum value, and a maximum value can be used as the representative value.

Further, the first evaluation value derivation unit 24 derives representative values of the diameters of the plurality of cross sections orthogonal to the central axis 36 in each of the head portion 33, the body portion 34, and the caudal portion 35 as the respective diameters of the head portion 33, the body portion 34, and the caudal portion 35. For example, an average value, a median value, a minimum value, and a maximum value of the diameters of the plurality of cross sections can be used as the representative value. The representative values of the diameters of the head portion 33, the body portion 34, and the caudal portion 35 are the first evaluation values E11, E12, and E13, respectively.

It should be noted that the first evaluation value derivation unit 24 may derive a representative value of an area in the plurality of cross sections or a volume of the head portion 33, the body portion 34, and the caudal portion 35 as the first evaluation value instead of the respective diameters of the head portion 33, the body portion 34, and the caudal portion 35. The area of the cross section can be the number of pixels in the cross section, and the volumes of the head portion 33, the body portion 34, and the caudal portion 35 can be the number of pixels in the head portion 33, the body portion 34, and the caudal portion 35.

In addition, in a case in which more than three small regions are set as illustrated in FIG. 6 and FIG. 7, the first evaluation value derivation unit 24 may derive a representative value of diameter, a representative value of area, or a volume for each of the plurality of small regions, as the first evaluation value.

The second evaluation value derivation unit 25 derives at least one second evaluation value representing a relationship between the first evaluation values E11, E12, and E13 in the plurality of small regions. In the present embodiment, the second evaluation value derivation unit 25 derives an evaluation value related to the difference in the size between the plurality of small regions as the second evaluation value. Specifically, the second evaluation value derivation unit 25 derives a ratio E11/E12 of a first evaluation value E11 for the head portion 33 to a first evaluation value E12 for the body portion 34 of the pancreas 30, and a ratio E12/E13 of the first evaluation value E12 for the body portion 34 to a first evaluation value E13 for the caudal portion 35 of the pancreas, as the second evaluation values E21, E22, respectively.

It should be noted that, instead of a ratio, an absolute value |E12-E11| of a difference between the first evaluation value E12 of the body portion 34 of the pancreas 30 and the first evaluation value E11 of the head portion 33 and an absolute value |E13-E12| of a difference between the first evaluation value E13 of the caudal portion 35 of the pancreas and the first evaluation value E12 of the body portion 34 may be derived as the second evaluation values E21, E22, respectively.

In addition, in a case in which more than three small regions are set as illustrated in FIG. 6 and FIG. 7, the second evaluation value representing the relationship between the first evaluation values in adjacent small regions may be derived. For example, the second evaluation value derivation unit 25 may derive the ratio between the first evaluation values derived for the plurality of small regions or the absolute value of the difference between the first evaluation values as the second evaluation value. The ratio may be, for example, the ratio of the first evaluation value of the small region on the left to the first evaluation value of the small region on the right in a case in which the human body is viewed from the front, but it may be the opposite.

It should be noted that not only the relationship between the first evaluation values in adjacent small regions but also the relationship between the first evaluation values in distant small regions may be derived. For example, in a case in which the pancreas 30 is divided into three small regions of the head portion 33, the body portion 34, and the caudal portion 35, a ratio E11/E13 of the first evaluation value E11 for the head portion 33 to the first evaluation value E13 for the caudal portion 35 or the absolute value |E11-E13| of the difference between the first evaluation values may be derived as the second evaluation value. In addition, in a case in which small regions are set as illustrated in FIG. 7, a ratio of the first evaluation value of a small region 37A to the first evaluation value of the small region 37E or an absolute value of a difference between the first evaluation values may be derived as the second evaluation value.

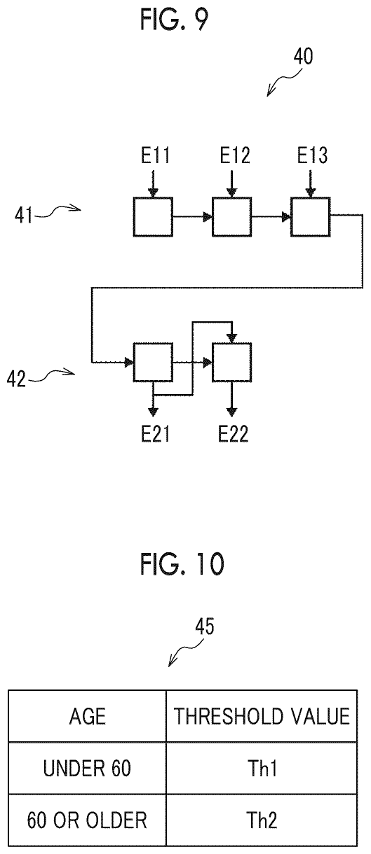

In addition, the second evaluation value derivation unit 25 may use a recurrent neural network (hereinafter, referred to as RNN) subjected to machine learning so as to output the second evaluation value in a case in which the plurality of first evaluation values are sequentially input along the central axis 36 of the pancreas 30. FIG. 9 is a diagram illustrating a schematic configuration of the RNN that derives the second evaluation value. As illustrated in FIG. 9, the RNN 40 consists of an encoder 41 and a decoder 42. The first evaluation values E11, E12, and E13 are sequentially input to three nodes constituting the encoder 41. The decoder 42 has been trained to derive the second evaluation value representing the relationship between the first evaluation values in adjacent small regions, and derives the second evaluation values E21 and E22 from the input first evaluation values E11, E12, and E13.

The third evaluation value derivation unit 26 derives a third evaluation value that indicates the presence or absence of an abnormality in the entire pancreas, which is the target organ, based on the second evaluation value. Here, in a case in which a tumor develops in the pancreas 30, various findings appear in the pancreas 30. For example, the pancreas parenchyma in the periphery of the tumor swells or the pancreas parenchyma other than the tumor undergoes the atrophy. In addition, a main pancreatic duct in the pancreas undergoes the dilation or the stenosis. In the present embodiment, the third evaluation value derivation unit 26 derives a probability of the atrophy of the pancreas as the third evaluation value based on the second evaluation values E21 and E22.

In the present embodiment, the third evaluation value derivation unit 26 includes a derivation model subjected to machine learning so as to derive the probability of the atrophy of the entire pancreas in a case in which the second evaluation values E21 and E22 are input. Similar to the SS model, the derivation model is configured by a convolutional neural network.

It should be noted that the derivation model of the third evaluation value derivation unit 26 may derive the presence or absence of the atrophy as the third evaluation value instead of the probability of the atrophy. In addition, the third evaluation value derivation unit 26 may compare the second evaluation values E21 and E22 with a predetermined threshold value, and in a case in which at least one of the second evaluation values E21 or E22 exceeds the threshold value, derive the determination result indicating that atrophy is present as the third evaluation value. In this case, in a case in which all the second evaluation values E21 and E22 are equal to or less than the threshold value, the third evaluation value derivation unit 26 derives the determination result indicating that the atrophy is absent as the third evaluation value. It should be noted that the presence or absence of the atrophy may be derived as the third evaluation value by comparing an addition value or a weighted addition value of the second evaluation values with the threshold value. It should be noted that the third evaluation value derivation unit 26 derives, for example, a value of 1 in a case in which the atrophy is present and a value of 0 in a case in which the atrophy is absent as the third evaluation value.

On the other hand, atrophy of the pancreas or dilation of the main pancreatic duct appears with aging. Therefore, a table in which a threshold value for determining the presence or absence of atrophy is defined according to the age may be prepared, and the third evaluation value may be derived with reference to the table. FIG. 10 is a diagram illustrating a table of a threshold value for deriving the third evaluation value. As illustrated in FIG. 10, a table 45 includes a threshold value Th1 used in a case in which the patient is under 60 years of age and a threshold value Th2 used in a case in which the patient is 60 years of age or older. Th1 is less than Th2. It should be noted that the table 45 may be stored in the storage 13.

In addition, in a case in which the probability of the atrophy is larger than a predetermined threshold value (for example, 0.6), or in a case in which the third evaluation value with atrophy is derived, the third evaluation value derivation unit 26 may specify the small region from which the largest second evaluation value among a plurality of second evaluation values is derived as the region that is the basis for the atrophy. For example, among the second evaluation values E21 and E22, the probability of the atrophy derived from the second evaluation value E22 is larger. In this case, since the second evaluation value E22 is the ratio of the first evaluation value E12 of the body portion 34 to the first evaluation value E13 of the caudal portion 35 of the pancreas, the third evaluation value derivation unit 26 specifies at least one of the body portion 34 or the caudal portion 35 as the region of that is the basis for the atrophy. It should be noted that a small region having a larger first evaluation value or a small region having a smaller first evaluation value in the body portion 34 and the caudal portion 35 may be specified as the region that is the basis for the atrophy. For example, in the present embodiment, the body portion 34 having the larger first evaluation value among the body portion 34 and the caudal portion 35, is specified as the region that is the basis for the atrophy.

It should be noted that, in a case in which more than three small regions are set as illustrated in FIG. 6 and FIG. 7, in addition to the small region from which the largest second evaluation value is derived, the small regions from which the second evaluation values up to the highest order in size are derived, such as second or third, may be specified as the region that is the basis for the atrophy.

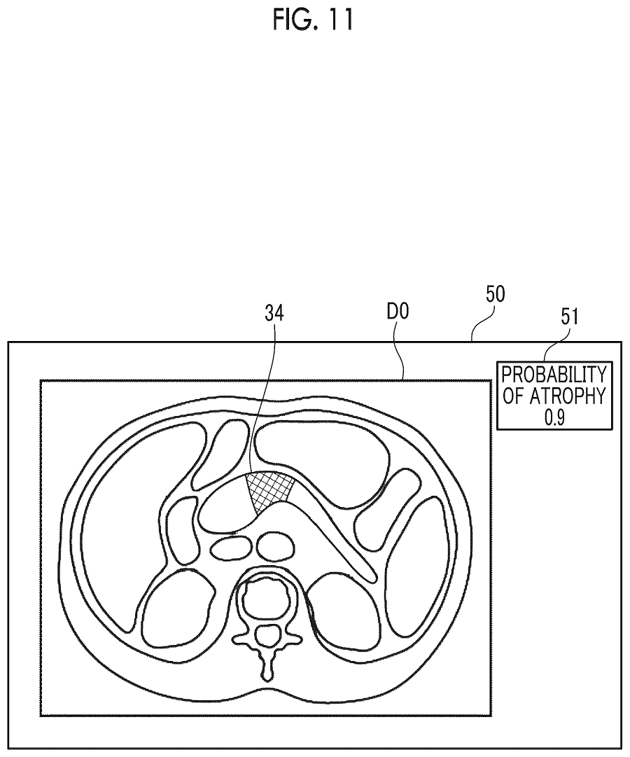

The display control unit 27 displays the evaluation result based on the third evaluation value on the display 14. FIG. 11 is a diagram illustrating a display screen of the evaluation result. As illustrated in FIG. 11, one tomographic image D0 of the target images G0 and an evaluation result 51 are displayed on an evaluation result display screen 50. In FIG. 11, the evaluation result 51 is the probability of the atrophy which is the third evaluation value itself. In FIG. 11, 0.9 is displayed as the probability of the atrophy.

In the present embodiment, a position of the small region having a relatively high contribution to the evaluation result based on the third evaluation value in the pancreas 30 is distinguished from a position of the small region having a relatively low contribution, and the tomographic image D0 is displayed. In the present embodiment, the third evaluation value derivation unit 26 specifies the body portion 34 as the region that is the basis for the atrophy. Therefore, the display control unit 27 displays the tomographic image D0 by distinguishing the body portion 34 from the head portion 33 and the caudal portion 35. Here, the body portion 34 is an example of a small region having a relatively high contribution to the evaluation result, and the head portion 33 and the caudal portion 35 are examples of a small region having a relatively low contribution to the evaluation result.

Specifically, as illustrated in FIG. 11, a region of the body portion 34 is displayed with more emphasis than the head portion 33 and the caudal portion 35. For example, the region of the body portion 34 may be highlighted and displayed by giving a color to the body portion 34. In FIG. 11, the fact that the body portion 34 of the pancreas 30 is colored is illustrated by giving hatching. It should be noted that the body portion 34 may be highlighted and displayed by giving a color to the outline of the body portion 34 or increasing the brightness. In addition, the highlighted display may be switched on or off by an instruction from the input device 15.

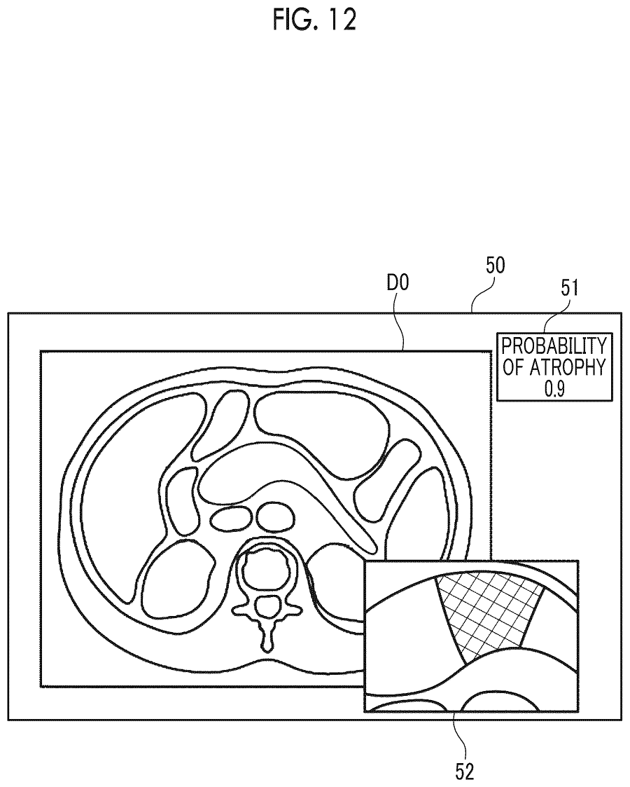

It should be noted that, by the instruction from the input device 15, a window 52 including an enlarged region of the pancreas 30 may be displayed in a separate window from the target image G0 (tomographic image D0), and the pancreas 30 displayed in the window 52 may be highlighted and displayed as illustrated in FIG. 12. Accordingly, it is possible to prevent the interpretation of the pancreas 30 from being hindered by the highlighted display.

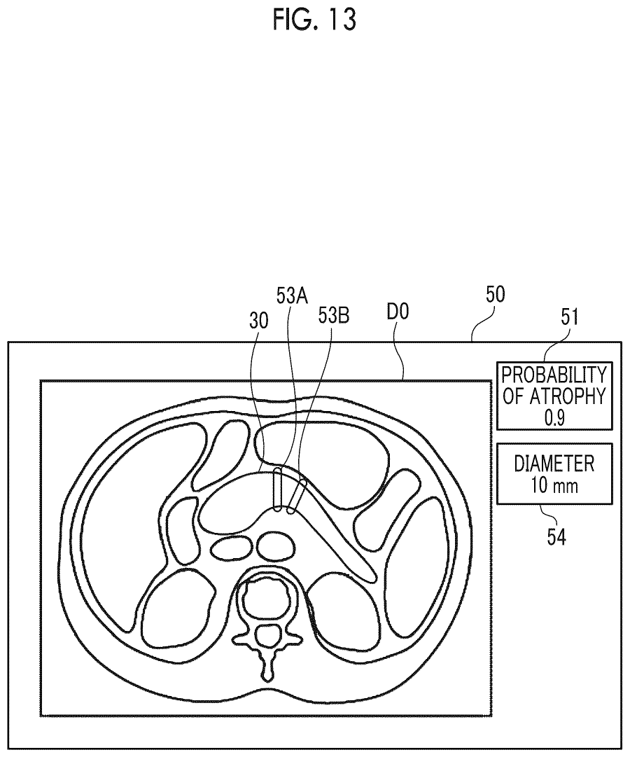

On the other hand, in a case in which more than three small regions are set as illustrated in FIG. 6 and FIG. 7, in the third evaluation value derivation unit 26, a plurality of small regions may be derived as the region that is the basis for the atrophy. In this case, as illustrated in FIG. 13, marks 53A and 53B may be given to the plurality of (two in FIG. 13) small regions. In addition, as illustrated in FIG. 13, a small region from which the smallest first evaluation value is derived among the small regions that are the basis for the atrophy may be specified, and a diameter of the small region 54 may be displayed as illustrated in FIG. 13. In FIG. 13, 10 mm is displayed as the diameter 54 of the smallest small region among the small regions that are the basis for the atrophy. It should be noted that the second evaluation value may be displayed in addition to the first evaluation value or instead of the first evaluation value.

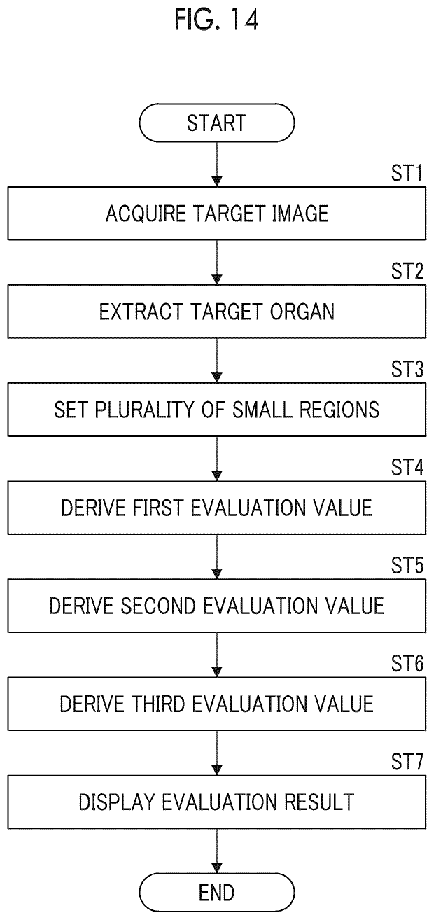

Hereinafter, processing performed in the present embodiment will be described. FIG. 14 is a flowchart illustrating the processing performed in the present embodiment. First, the image acquisition unit 21 acquires the target image G0 from the storage 13 (Step ST1), and the target organ extraction unit 22 extracts the pancreas, which is the target organ, from the target image G0 (Step ST2).

Next, the small region setting unit 23 sets a plurality of small regions in the pancreas which is the target organ (Step ST3). Then, the first evaluation value derivation unit 24 derives a first evaluation value representing a physical quantity in each of the plurality of small regions (Step ST4). Further, the second evaluation value derivation unit 25 derives at least one second evaluation value representing a relationship between the first evaluation values in the plurality of small regions (Step ST5), and the third evaluation value derivation unit 26 derives a third evaluation value indicating presence or absence of an abnormality in the entire pancreas 30, which is target organ, based on the second evaluation value (Step ST6). Then, the display control unit 27 displays an evaluation result on the display 14 based on the third evaluation value (Step ST7), and the processing ends.

As described above, in the present embodiment, a first evaluation value representing a physical quantity of each of the plurality of small regions is derived, at least one second evaluation value representing a relationship between the first evaluation values in the plurality of small regions is derived, and a third evaluation value indicating presence or absence of an abnormality in the entire target organ is derived based on the second evaluation value. Further, the evaluation result based on the third evaluation value is displayed on the display 14. Therefore, it is possible to accurately evaluate an abnormality of the target organ in consideration of a relationship between the plurality of small regions in the target organ.

In addition, by using the first evaluation value as a physical quantity related to the size of the small region and the second evaluation value as an evaluation value related to the size difference between small regions, it is possible to accurately evaluate an abnormality caused by size changes such as atrophy and swelling in the target organ.

In addition, by setting an axis passing through the target organ and setting the target organ along the axis, it is possible to easily set the small region for the target organ having an elongated shape, such as the pancreas.

In addition, by displaying a position of the small region having a relatively high contribution to the evaluation result in the target organ on the display 14 as distinguished from a position of the small region having a relatively low contribution, it becomes easy to recognize the position of the small region having a relatively high contribution to the evaluation result. Therefore, it is possible to easily specify a position where an abnormality is likely to be present in the target organ.

It should be noted that, in the embodiment described above, the third evaluation value related to the atrophy of the pancreas is derived as a finding, but the present disclosure is not limited to this. The third evaluation value related to the swelling of the pancreas may be derived. In this case, the third evaluation value derivation unit 26 may derive the probability of the swelling or the presence or absence of the swelling as the third evaluation value.

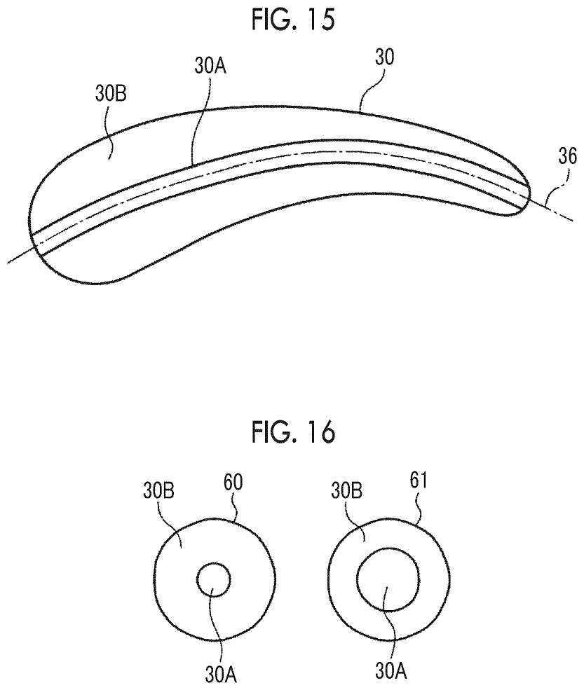

In addition, in the embodiment described above, the plurality of small regions are set along the longitudinal direction of the pancreas, but the present disclosure is not limited to this. Here, as illustrated in FIG. 15, in the pancreas 30, a main pancreatic duct 30A is present along the central axis 36 of the pancreas 30. In the CT image, since the CT values are different between the main pancreatic duct 30A and the pancreas parenchyma 30B, the pancreas 30 can be divided into a region of the main pancreatic duct 30A and a region of the pancreas parenchyma 30B. Therefore, by dividing the pancreas 30 into the main pancreatic duct 30A and the pancreas parenchyma 30B, the main pancreatic duct 30A and the pancreas parenchyma 30B may be set as small regions, respectively.

In this case, a diameter of the main pancreatic duct 30A and a diameter of the pancreas parenchyma 30B may be derived as the first evaluation values. The diameters of the main pancreatic duct 30A and the pancreas parenchyma 30B may be obtained by dividing the main pancreatic duct 30A and the pancreas parenchyma 30B into the plurality of small regions along the central axis 36 of the pancreas 30 and deriving the diameters of the main pancreatic duct 30A and the pancreas parenchyma 30B in the small regions in the same manner as the diameters of the head portion 33, the body portion 34, and the caudal portion 35 are derived. In this case, as the second evaluation value, a ratio of the diameter of the main pancreatic duct 30A to the diameter of the pancreas parenchyma 30B, or an absolute value of a difference between the diameters can be used.

In addition, small regions may be set by dividing only one of the main pancreatic duct 30A or the pancreas parenchyma 30B into the plurality of regions along the central axis 36 of the pancreas 30, and the first evaluation value may be derived for each small region. In particular, in a case in which the plurality of small regions are set only in the main pancreatic duct 30A, the first evaluation value for the plurality of small regions is derived and at least one second evaluation value representing the relationship between the first evaluation values in the plurality of small regions is derived. In this case, the dilation or the stenosis of the main pancreatic duct 30A can be specified as a finding using the second evaluation value. Therefore, based on the second evaluation value, it is possible to derive the third evaluation value indicating the presence or absence of the dilation or the stenosis of the main pancreatic duct 30A as a finding.

In addition, the ratio of the diameter of the main pancreatic duct 30A to the diameter of the pancreas parenchyma 30B or the absolute value of the difference between the diameters may be derived as the second evaluation value in each of the head portion 33, the body portion 34, and the caudal portion 35 of the pancreas 30. FIG. 16 is a diagram schematically illustrating a cross section orthogonal to the central axis 36 of the pancreas 30. It should be noted that both the cross section of the pancreas 30 and the cross section of the main pancreatic duct 30A are illustrated by circles in FIG. 16. A cross section 60 and a cross section 61 illustrated in FIG. 16 represent cross sections in the adjacent small regions. In the cross section 60 illustrated in FIG. 16, the ratio of the diameter of the main pancreatic duct 30A to the diameter of the pancreas 30 is about 0.2, and in the cross section 61, the ratio of the diameter of the main pancreatic duct 30A to the diameter of the pancreas 30 is about 0.5. In this case, the second evaluation value is 0.3 in a case in which the second evaluation value is an absolute value of a difference between the first evaluation values. Such a second evaluation value is an indicator indicating the dilation or the stenosis of the main pancreatic duct 30A. Therefore, for example, in a case in which the third evaluation value is derived by setting the threshold value to 0.1, the third evaluation value indicating that the dilation of the pancreatic duct is present can be derived in the case of the cross sections 60 and 61 as illustrated in FIG. 16.

In addition, in the embodiment described above, as the first evaluation value, a physical quantity related to the size of the small region such as the diameter, the area, and the volume of the small region is used, but the present disclosure is not limited to this. The physical quantity related to a property of the small region may be used as the first evaluation value. For example, in a case in which CT values are compared along the direction of the central axis 36 in the pancreas 30, if there is a place where the CT value changes rapidly, there is a high probability of an abnormality occurring in that place. Therefore, a representative value of the CT values may be derived as the first evaluation value for each of the plurality of small regions. For example, an average value, a median value, a dispersion value, a minimum value, and a maximum value can be used as the representative value. In this case, as the second evaluation value, the ratio of the first evaluation values in the adjacent small regions or the absolute value of the difference between the first evaluation values can be used.

In addition, a feature map consisting of an image of the small region itself of a feature amount derived from the image of the small region may be used as the first evaluation value. The feature amount can be derived by performing filtering processing on the image of the small region with a filter having a predetermined size with a predetermined filter coefficient.

In addition, while the pancreas has an oval shape in a cross section orthogonal to the central axis thereof, the shape of the cross section is distorted in a case in which an abnormality occurs. Therefore, a shape value such as the roundness of the small region may be used as the physical quantity related to the property of the small region which is the first evaluation value. As the shape value, the roundness of each of the head portion 33, the body portion 34, and the caudal portion 35 of the pancreas 30 can be used. Here, in a case in which the central axis 36 of the pancreas 30 is set as illustrated in FIG. 6, the shape of the cross section orthogonal to the central axis 36 is an oval shape in a portion without the abnormality. For this reason, the first evaluation value derivation unit 24 derives lengths in a plurality of directions (for example, four directions of the up-down direction, the left-right direction, the lower right to upper left direction, and the lower left to upper right direction) passing through the central axis 36 in the same manner as a case of obtaining the diameter illustrated in FIG. 8, and derives a value of ½ of a difference between the maximum value and the minimum value of the lengths in the plurality of directions as a roundness.

Further, the first evaluation value derivation unit 24 derives a representative value of the roundness of the plurality of cross sections orthogonal to the central axis 36 in each of the head portion 33, the body portion 34, and the caudal portion 35 as the respective roundness of the head portion 33, the body portion 34, and the caudal portion 35. As the representative value, an average value, a median value, a dispersion value, a minimum value, and a maximum value of the roundness of the plurality of cross sections can be used. The representative values of the roundness of the head portion 33, the body portion 34, and the caudal portion 35 are the first evaluation values, respectively.

Here, the roundness of the cross section of the portion at which the abnormality has occurred in the pancreas is smaller than the roundness of the cross section of the normal portion. Therefore, by using the roundness as the first evaluation value and deriving, as the second evaluation value, the ratio of the first evaluation values in the adjacent small regions or the absolute value of the difference between the first evaluation values, it is possible to derive a third evaluation value indicating the presence or absence of the abnormality in the pancreas

In addition, in the embodiment described above, the target organ is the pancreas, but the present disclosure is not limited to this. In addition to the pancreas, any organ, such as the brain, the heart, the lung, and the liver, can be used as the target organ.

In addition, in the embodiment described above, the CT image is used as the target image G0, but the present disclosure is not limited to this. In addition to the three-dimensional image, such as the MRI image, any image, such as a radiation image acquired by simple imaging, can be used as the target image G0.

In addition, in the embodiment described above, various processors shown below can be used as the hardware structure of the processing units that execute various types of processing, such as the image acquisition unit 21, the target organ extraction unit 22, the small region setting unit 23, the first evaluation value derivation unit 24, the second evaluation value derivation unit 25, the third evaluation value derivation unit 26, and the display control unit 27. As described above, the various processors include, in addition to the CPU that is a general-purpose processor which executes software (program) to function as various processing units, a programmable logic device (PLD) that is a processor of which a circuit configuration can be changed after manufacture, such as a field programmable gate array (FPGA), and a dedicated electrical circuit that is a processor having a circuit configuration which is designed for exclusive use to execute a specific processing, such as an application specific integrated circuit (ASIC).

One processing unit may be configured by one of these various processors or may be configured by a combination of two or more processors of the same type or different types (for example, a combination of a plurality of FPGAs or a combination of the CPU and the FPGA). In addition, a plurality of processing units may be formed of one processor.

As an example of configuring the plurality of processing units by one processor, first, as represented by a computer of a client, a server, and the like, there is an aspect in which one processor is configured by a combination of one or more CPUs and software and this processor functions as a plurality of processing units. Second, as represented by a system on chip (SoC) or the like, there is an aspect of using a processor that realizes the function of the entire system including the plurality of processing units by one integrated circuit (IC) chip. In this way, as the hardware structure, the various processing units are configured by using one or more of the various processors described above.

Further, as the hardware structures of these various processors, more specifically, it is possible to use an electrical circuit (circuitry) in which circuit elements, such as semiconductor elements, are combined.

What is claimed is:

1. An image processing apparatus comprising:
at least one processor,
wherein the processor is configured to:
extract a target organ from a medical image;
set a plurality of small regions in the target organ;
derive a first evaluation value representing a physical quantity of each of the plurality of small regions;
derive at least one second evaluation value representing a relationship between the first evaluation values in the plurality of small regions; and
derive a third evaluation value indicating presence or absence of an abnormality in the entire target organ based on the second evaluation value.

2. The image processing apparatus according to claim 1,
wherein the first evaluation value is a physical quantity related to a size of the small region, and
the second evaluation value is an evaluation value related to a difference in sizes between the small regions.

3. The image processing apparatus according to claim 1,
wherein the processor is configured to:
set an axis passing through the target organ; and
set the small region along the axis.

4. The image processing apparatus according to claim 2,
wherein the processor is configured to:
set an axis passing through the target organ; and
set the small region along the axis.

5. The image processing apparatus according to claim 1,
wherein the processor is configured to display an evaluation result based on the third evaluation value on a display.

6. The image processing apparatus according to claim 5,
wherein the evaluation result based on the third evaluation value is an occurrence probability of a finding representing a feature of a shape of the target organ.

7. The image processing apparatus according to claim 6,
wherein the finding includes at least one of atrophy, swelling, stenosis, or dilation that occurs in the target organ.

8. The image processing apparatus according to claim 5,
wherein the processor is configured to display a position of the small region having a relatively high contribution to the evaluation result in the target organ on the display as distinguished from a position of the small region having a relatively low contribution.

9. The image processing apparatus according to claim 6,
wherein the processor is configured to display a position of the small region having a relatively high contribution to the evaluation result in the target organ on the display as distinguished from a position of the small region having a relatively low contribution.

10. The image processing apparatus according to claim 7,
wherein the processor is configured to display a position of the small region having a relatively high contribution to the evaluation result in the target organ on the display as distinguished from a position of the small region having a relatively low contribution.

11. The image processing apparatus according to claim 8,
wherein the processor is configured to display at least one of the first evaluation value or the second evaluation value of the small region having the relatively high contribution to the evaluation result in the target organ on the display.

12. The image processing apparatus according to claim 9,
wherein the processor is configured to display at least one of the first evaluation value or the second evaluation value of the small region having the relatively high contribution to the evaluation result in the target organ on the display.

13. The image processing apparatus according to claim 10,
wherein the processor is configured to display at least one of the first evaluation value or the second evaluation value of the small region having the relatively high contribution to the evaluation result in the target organ on the display.

14. The image processing apparatus according to claim 1,
wherein the medical image is a tomographic image of an abdomen including a pancreas, and
the target organ is a pancreas.

15. The image processing apparatus according to claim 14,
wherein the processor is configured to set the small region by dividing the pancreas into a head portion, a body portion, and a caudal portion.

16. An image processing method comprising:
extracting a target organ from a medical image;
setting a plurality of small regions in the target organ;
deriving a first evaluation value representing a physical quantity of each of the plurality of small regions;
deriving at least one second evaluation value representing a relationship between the first evaluation values in the plurality of small regions; and
deriving a third evaluation value indicating presence or absence of an abnormality in the entire target organ based on the second evaluation value.

17. A non-transitory computer-readable storage medium that stores an image processing program causing a computer to execute:
a procedure of extracting a target organ from a medical image;
a procedure of setting a plurality of small regions in the target organ;
a procedure of deriving a first evaluation value representing a physical quantity of each of the plurality of small regions;
a procedure of deriving at least one second evaluation value representing a relationship between the first evaluation values in the plurality of small regions; and
a procedure of deriving a third evaluation value indicating presence or absence of an abnormality in the entire target organ based on the second evaluation value.
